(19) United States
(12) Patent Application Publication
(10) Pub. No.: US 2026/0000712 A1
(43) Pub. Date: Jan. 1, 2026

(54) METHOD FOR MODULATING GLUCAGON-LIKE PEPTIDE 1 (GLP-1) AGONIST INDUCED MUSCLE LOSS

(71) Applicant: Celularity Inc., Florham Park, NJ (US)

(72) Inventors: Sharmila KOPPISETTI, Florham Park, NJ (US); Robert J. HARIRI, Florham Park, NJ (US); Joseph GLEASON, Florham Park, NJ (US); Rajarajeswari SIVALENKA, Florham Park, NJ (US); Lin KANG, Florham Park, NJ (US); Anna GOSIEWSKA, Florham Park, NJ (US)

(73) Assignee: Celularity Inc., Florham Park, NJ (US)

(21) Appl. No.: 19/094,351

(22) Filed: Mar. 28, 2025

Related U.S. Application Data
(60) Provisional application No. 63/571,116, filed on Mar. 28, 2024.

(51) Int. Cl. A61K 35/28

(52) U.S. Cl. CPC A61K 35/28

(57) ABSTRACT

Provided herein is a method of ameliorating muscle loss in a subject induced from the treatment of the subject with a GLP-1 agonist with the administration to the subject of a therapeutically effective amount of a placenta derived biological material having a continuous lipid bilayer membrane

### PRIORITY CLAIM

This application claims priority to U.S. Provisional Application 63/571,116 filed 28 Mar. 2024, which is hereby incorporated by reference in its entirety.

### FIELD OF INVENTION

The instant disclosure relates generally to a method of modulating muscle loss in a subject that is induced due to the subject's treatment with a GLP-1 agonist.

### BACKGROUND

Glucagon-like peptide-1 (GLP-1) agonists (also known as GLP-1 receptor agonists, incretin mimetics, or GLP-1 analogs) represent a class of medications used to treat type 2 diabetes mellitus and used for weight loss. Examples of drugs in this class include exenatide, lixisenatide, liraglutide, albiglutide, dulaglutide, and semaglutide, to name only a few. This class of medications has also been shown to promote an average weight loss of 2.9 kilograms compared to placebo, in addition to lowering both systolic and diastolic blood pressure and total cholesterol. Other functions of GLP-1 agonists include increased glucose uptake in the muscles, decreased glucose production in the liver, neuroprotection, and increased satiety due to direct actions on the hypothalamus (https://www.ncbi.nlm.nih.gov/books/NBK551568/).

Studies have demonstrated that when body weight (BW) is reduced through dietary energy restriction, not all the resulting weight loss can be attributed to fat mass (FM). It has been determined that approximately 25% to 33% of such reduction in BW is estimated to comprise reductions in lean body mass (LBM), which is predominantly comprised of skeletal muscle. Skeletal muscle acts as a primary site of glucose disposal (with lower skeletal muscle mass contributing to poorer glycemic control and is a strong determinant of resting metabolic rate). Thus, loss of skeletal muscle with weight loss may predispose individuals to a greater chance of weight regain. Lower muscle mass and function, associated with impaired muscular strength and endurance, also results in a higher risk of falls, hospitalization, and physical issues (http://www.ncbi.nlm.nih.gov/pmc/articles/PMC6769337/).

Recent research on glucagon-like peptide-1 (GLP-1) drugs for weight loss indicates these medications may lead to significant reductions in lean body mass, including muscle. The STEP 1 and SUSTAIN 8 trials of semaglutide trials found 39-40% of weight lost on the GLP-1 drugs was lean mass. A 2021 meta-analysis of 18 randomized controlled studies showed significant drops in fat-free mass with GLP-1 agonists, including oral and subcutaneous semaglutide and older GLP-1 agonists such as lixisenatide, exenatide, and liraglutide (https://www.drugdiscoverytrends.com/glp-1-impact-lean-mass/#:˜:text=Yes%2C%20recent%20research%20on%20glucagon,1%20drugs %20was%20lean%20mass). Per CDC, 41.5% of older U.S. adults have obesity (https://www.cdc.gov/obesity/data/adult.html#:˜:text=The%20obesity%20prevalence%20was%2039.8,adults%20and%2060%20and%20older) and could benefit from a weight loss medication. However, this older population may experience additional loss of muscle mass when taking a GLP-1 RA medication for the treatment of obesity.

Accordingly, what is needed is a pharmaceutical composition that can modulate, and in particular, retard, halt, or ameliorate muscle loss in patients being treated with GLP-1 agonists.

The citation of any reference herein should not be deemed as an admission that such reference is available as prior art to the instant disclosure.

### SUMMARY OF THE DISCLOSURE

The present disclosure extends to a method for modulating Glucagon-Like Peptide 1 (GLP-1) agonist induced muscle loss in a subject being treated with a GLP-1 agonist, comprising administrating to the subject a therapeutically effective amount of a placenta derived biological material having a continuous lipid bilayer membrane, an umbilical cord derived biological material having a continuous lipid bilayer membrane, or a combination thereof.

Such modulation includes, but is not limited to slowing down, or retarding GLP-1 agonist induced muscle loss in the subject, stopping or arresting such GLP-1 agonist induced muscle loss in the subject, or ameliorating muscle loss in the subject.

Moreover, a number of biological materials having a lipid bilayer have applications in a method of the instant disclosure. One example of such material is placenta derived adherent cells, or a population thereof. Another example of such biological material is exosomes. In a particular embodiment, such exosomes are secreted from placenta derived adherent cells or a population thereof. Such secretion can occur in vivo, in vitro, ex vivo, or any combination thereof. Naturally, a biological material having applications in a method of the instant disclosure includes a population of placenta derived adherent cells or a population thereof combined with exosomes secreted from the placenta derived adherent cells or a population thereof. Particular placenta derived adherent cells having applications herein include, but certainly are not limited to, PDA-001, PDA-002, or a combination thereof. Yet another placenta derived biological material having a continuous lipid bilayer lipid membrane is secretomes. Such secretomes can readily be isolated from placenta or umbilical cord. In a particular embodiment, such secretomes contain exosomes.

Likewise, a number of biological materials having a lipid that have applications herein can be derived from umbilical cord. In a particular embodiment, such a biological material is secretomes that contain exosomes.

Furthermore, in a method of the instant disclosure, the subject is being treated with a GLP-1 agonist for a variety of reasons. Examples include but certainly are not limited to the treatment of type II diabetes, for weight loss, or a combination thereof. Muscle loss induced by a number of GLP-1 agonists can be treated with a method of the instant disclosure. Examples of such GLP-1 agonists include dulaglutide, exenatide, exenatide extended-release, liraglutide, dulaglutide, semaglutide injection, semagulutide tablet, lixisenatide, albiglutide, tirzepatide, and efpeglenatide. Furthermore, the mode of administration of a therapeutically effective amount of a placenta derived biological material having a continuous lipid bilayer membrane, an umbilical cord derived biological material having a continuous lipid bilayer membrane, or a combination thereof can vary and includes intradermal administration, subcutaneous administration, intramuscular administration, and intravenous administration. In a particular embodiment, the administration is transmuscular administration.

The instant disclosure further extends to a method for modulating GLP-1 agonist induced muscle loss in a subject being treated with a GLP-1 agonist, comprising the administration to the subject of a therapeutically effective amount of a population of placenta derived adherent cells, such as PDA-001 cells, PDA-002 cells, or a combination thereof.

Yet another embodiment of the instant disclosure is a method for modulating GLP-1 agonist induced muscle loss in a subject being treated with a GLP-1 agonist, comprising the administration to the subject of a therapeutically effective amount of placenta derived secretomes. In a particular embodiment, such secretomes contain exosomes.

Also provided in the instant disclosure is a method for modulating GLP-1 agonist induced muscle loss in a subject being treated with a GLP-1 agonist, comprising the administration to the subject of a therapeutically effective amount of exosomes secreted from placenta derived adherent cells. In a particular embodiment, the exosomes are secreted from PDA-001 cells or PDA-002 cells.

The instant disclosure further extends to a method for modulating GLP-1 agonist induced muscle loss in a subject being treated with a GLP-1 agonist, comprising the administration to the subject of a therapeutically effective amount of a placenta derived biological material having a continuous lipid bilayer, wherein the placenta derived biological material comprises a combination of a population of placenta derived adherent cells, e.g. PDA-001 cells, PDA-002 cells, or a combination thereof, and exosomes secreted from PDA-001 cells, PDA-002 cells, or secreted from a combination of PDA-001 and PDA-002 cells.

Moreover, the instant disclosure extends to a pharmaceutical composition comprising a placenta derived biological material having a continuous lipid bilayer membrane as described herein and a pharmaceutically acceptable excipient or carrier.

These and other aspects of the present disclosure will be better appreciated by reference to the following drawings and Detailed Description.

### DESCRIPTION OF THE FIGURES

FIG. 1: Distribution of Body Weights During the Study-PDA-001.

FIG. 2: Blood Flow Data During the Study-PDA-001.

FIG. 3: Blood Volume of the Different Groups Through the Study Measured by Ultrasound Image Analysis-PDA-001.

FIG. 4: Blood Flow Velocity of the Different Groups Through the Study Measured by Ultrasound Image Analysis-PDA-001.

FIG. 5: Blood Flow Ratio of the Different Groups Through the Study Measured by Ultrasound Image Analysis-PDA-001.

FIG. 6: Hind Limb Ischemic Severities of the Groups through the Study (Last Observation analysis)-PDA-001.

FIG. 7: Hind Limb Amputation Dynamics by Groups Through the Study (KaplanMayer Curve Analysis)-PDA-001

FIG. 8: Toe Necrosis Dynamics by Groups Through the Study (Kaplan-Mayer Curve Analysis)-PDA-001.

FIG. 9: Hind Limb Functions of the Groups Through the Study (Last Observation Analysis)-PDA-001.

### DETAILED DESCRIPTION

Broadly, the present disclosure extends to a method for modulating Glucagon-Like Peptide 1 (GLP-1) agonist induced muscle loss in a subject being treated with a GLP-1 agonist, comprising administrating to the subject a therapeutically effective amount of a placenta derived biological material having a continuous lipid bilayer membrane, an umbilical cord derived biological material having a continuous lipid bilayer membrane, or a combination thereof.

Such modulation includes, but is not limited to slowing down, or retarding GLP-1 agonist induced muscle loss in the subject, stopping or arresting such GLP-1 agonist induced muscle loss in the subject, or ameliorating muscle loss in the subject.

Unless defined otherwise, all technical and scientific terms used herein have the same meanings as commonly understood by one of ordinary skill in the art to which this disclosure belongs. Although any methods and materials similar or equivalent to those described herein can be used in the practice or testing of the present disclosure.

All numerical designations, e.g. volume, mass, etc., are approximations which are varied by (+) or (−) by increments of 1.0 or 0.1, as appropriate. It is to be understood, although not always explicitly stated, that all numerical designations are preceded by the term “about”.

Numerous terms and phrases are used throughout the instant specification and claims and are defined below.

“About” and “approximately” are interchangeable and mean plus or minus a percent (e.g., ±5%) of the number, parameter, or characteristic so qualified, which would be understood as appropriate by a skilled artisan to the scientific context in which the term is utilized.

As used herein, the singular form “a,” “an” and “the” include plural reference unless the context clearly dictates otherwise.

As used herein, the terms “comprising,” “comprises” and “comprise” are intended to mean that the compositions, preparations and methods disclosed herein include recited elements, but do not exclude others.

Therefore, if appearing herein, the following terms and phrases shall have the definitions set out below.

As used herein, the phrase “Glucagon-Like Peptide 1” (GLP-1) refers to a hormone having metabolic and insulinotropic effects. GLP-1 stimulates insulin secretion from pancreatic β-cells and inhibits glucagon secretion from pancreatic α-cells (Ørskov, et al., Diabetes, 42:658-61, 1993; D'Alessio, et al., J. Clin. Invest., 97:133-38, 1996), GLP-1 is reported to inhibit gastric emptying (Willms B, et al., J. Clin. Endocrinol. Metab. 81 (1): 327-32, 1996; Wettergren A, et al., Dig Dis Sci 38 (4): 665-73, 1993), and gastric acid secretion. Schjoldager B T, et al., Dig Dis Sci 34 (5): 703-8, 1989; O'Halloran D J, et al., J Endocrinol 126 (1): 169-73, 1990; Wettergren A, et al., Dig Dis sci 38 (4): 665-73, 1993). GLP-1 [7-37], [SEQ. ID. NO. 37] which has an additional glycine residue at its carboxy terminus, also stimulates insulin secretion in humans (Ørskov, et al., Diabetes, 42:658-61, 1993).

As used herein, the term “agonist” refers to a substance that binds to a receptor and activates the receptor to exhibit the same action as the receptor would exhibit when bound to its ligand. Thus, a GLP-1 agonist is a substance that binds to the same receptors to which GLP-1 binds and elicits the same response from the receptor as GLP-1 elicits. Examples of GLP-1 agonists include, but certainly are not limited to dulaglutide, exenatide, exenatide extended-release, liraglutide, dulaglutide, semaglutide injection, semagulutide tablet, lixisenatide, albiglutide, tirzepatide, and efpeglenatide, to name only a few. GLP-1 agonists may be used for the following medical treatments:
(i) prevention and/or treatment of all forms of diabetes, such as hyperglycemia, type 2 diabetes, impaired glucose tolerance, type 1 diabetes, non-insulin dependent diabetes, MODY (maturity onset diabetes of the young), gestational diabetes, and/or for reduction of HbA1c;
(ii) delaying or preventing diabetic disease progression, such as progression in type 2 diabetes, delaying the progression of impaired glucose tolerance (IGT) to insulin requiring type 2 diabetes, and/or delaying the progression of non-insulin requiring type 2 diabetes to insulin requiring type 2 diabetes;
(iii) prevention and/or treatment of eating disorders, such as obesity, e.g., by decreasing food intake, reducing body weight, suppressing appetite, inducing satiety; treating or preventing binge eating disorder, bulimia nervosa, and/or obesity induced by administration of an antipsychotic or a steroid; reduction of gastric motility; and/or delaying gastric emptying.

As used herein, the phrase “a continuous lipid bilayer membrane” refers to a thin polar membrane made of two layers of lipid molecules. These membranes are flat sheets that form a continuous barrier. They keep ions, proteins and other molecules localized and prevent them from diffusing into areas where they should not be. Continuous lipid bilayers are ideally suited to this role, even though they are only a few nanometers in width because they are impermeable to most water-soluble (hydrophilic) molecules and ions. Particular examples of continuous lipid bilayer membranes are cell membranes, including the membranes of placenta derived adherent cells, and vesicle membranes, such as for example an exosome membrane or a secretome membrane.

As used herein, the terms “carrier” and “excipient”, which can be used interchangeably, mean an inactive substance that serves as the vehicle or medium for a drug or other active substance. Numerous types of carriers have applications in the instant disclosure. For example, pharmaceutical compositions as disclosed herein may be formulated in a conventional manner using one or more physiologically acceptable carriers. Formulation of a pharmaceutical composition of the instant disclosure is dependent upon the desired route of administration. A summary of carriers having applications in a pharmaceutical composition described herein may be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins; 1999), all of which are hereby incorporated by reference in their entireties. Examples include but certainly are not limited to normal saline, a physiological salt solution (phosphate buffered saline; PBS), Dulbecco's Modified Eagle Solution (DMEM), water, any autologous preparation (such as platelet rich plasma (PRP), bone marrow aspirate concentrate (BMAC), stromal vascular fraction (SVF)), or a balanced salt solution (BSS), to name only a few. A pharmaceutically acceptable excipient having applications in a pharmaceutical composition of the instant disclosure can be a liquid or a solid.

As used herein, the term “placenta” means a temporary organ that forms in the uterus of a female during pregnancy, which attaches to the urine wall and provides nutrients and oxygens to the fetus through the umbilical cord. Parts of the placenta include chorion, amnion, chorionic villi, decidua basalis, chorion frondosum. For purposes of the instant disclosure the umbilical cord is not part of the placenta

As used herein the term “placenta-derived” with respect to a placenta derived biological material having a continuous lipid bilayer membrane, e.g., a cell, exosome or secretome, means that the source of the biological material is the placenta, which is typically obtained from animals (such as pigs or sheep), and in a particular embodiment, human placenta.

As used herein, the phrase “placenta derived adherent cells” (PDA cells) refers to culture expanded, undifferentiated mesenchymal-like cells derived from full-term placental tissue, which display immunomodulatory, anti-inflammatory, pro-regenerative, angiogenic, and neuroprotective properties The cells are anchorage-dependent in culture and will adhere to the plastic surface during expansion culture in vitro. Examples of PDA cells having applications herein are PDA-001 and PDA-002 cells. PDA-001 cells are PDA cells comprising CD44+, CD73+, CD90+, CD105+, CD200+, and CD45-PDA cells. PDA-002 cells are a culture expanded, undifferentiated mesenchymal-like cell population derived from normal, full-term human placental tissue with the genetic makeup of the baby, not the mother. These cells express the nominal phenotype CD34-, CD10+, CD105+, and CD200+, and constitutively express moderate levels of human leukocyte antigen (HLA) Class I and undetectable levels of HLA Class II.

As used herein, the term “umbilical cord” refers to a flexible cordlike structure containing blood vessels and attaching a human or other mammalian fetus to the placenta during gestation. As used herein, the phrase “umbilical cord derived biological material having a continuous lipid bilayer membrane” means such biological material is isolated from umbilical cord, particularly human umbilical cord.

As used herein, the term “exosome” refers to naturally existing nanoparticles that are secreted endogenously by many cell types, and are commonly found in vivo in body fluids, such as blood, urine and malignant ascites. Exosomes are cup-like multivesicular bodies (MVBs) varying in size between 30-100 nm, and contain various molecules such as proteins, lipids, and nucleic acids that can be transferred to other cells to influence their function.

As used herein, the term “secretome” means a collection of biomolecules, including proteins, lipids, nucleic acids, and extracellular vesicles, that are actively secreted by cells into their extracellular environment. Secretomes contain a broader range of molecules that are released from cells than are contained in exosomes.

As used herein, the term “modulating” with respect to GLP-1 agonist induced muscle loss means to alter the rate of muscle loss a subject exhibits due to the administration to the subject of a GLP-1 agonist. Modulation includes arresting or stopping induced muscle loss, slowing or retarding induced muscle loss, and ameliorating induced muscle loss.

As used herein, the phrase “therapeutically effective amount” refers to an amount of a therapeutic composition sufficient to produce a measurable biological response. Actual dosage levels of a placenta derived biological material having a continuous lipid bilayer membrane, e.g., placenta derived adherent cells such as, for example, PDA-001 and PDA-002 cells, exosomes, or secretomes, can be varied so as to administer an amount that is effective to achieve the desired therapeutic response for a particular subject and/or application. Of course, the therapeutically effective amount in any particular case will depend upon a variety of factors including the activity of the therapeutic composition, formulation, the route of administration, combination with other drugs or treatments, severity of the condition being treated, and the physical condition and prior medical history of the subject being treated. Preferably, a minimal dose is administered, and the dose is escalated in the absence of dose-limiting toxicity to a minimally effective amount. Determination and adjustment of a therapeutically effective dose, as well as evaluation of when and how to make such adjustments, are known to those of ordinary skill in the art.

The present disclosure may be better understood by reference to the following non-limiting examples, which are provided as exemplary of the instant disclosure. The following examples are presented in order to more fully illustrate particular embodiments of the disclosure. They should in no way be construed, however, as limiting the broad scope of the disclosure.

### EXAMPLES

### I. Exosomes

Exosomes (which includes extracellular vesicles (EVs)) are generated from late endosomes and micro-vesicles originating from the budding of the cell membrane, when fused with the targeted cells. The molecular cargos (proteins, lipids, DNA, mRNA, and microRNAs) they carry are then inserted into the cells to exert the functions of the exosomes. Recently, exosomes are being recognized as promising candidates in regenerative because evidence has suggested that part of the observed cell therapeutic effects is mediated by paracrine factors including exosomes secreted from the cells. EV-based therapeutics have certain advantages over cell therapy such as: low/non-immunogenicity, easy storage, and administration. In addition, due to their nano-size, EVs can cross the brain-blood barrier and can be delivered to broader target tissues and organs than cell-based therapeutics.

Accumulating evidence shows that MSC-derived exosomes extensively participate in the physiology and pathology of skeletal muscle.

Nakamura, Y. et al. Mesenchymal-stem-cell-derived exosomes accelerate skeletal muscle regeneration. FEBS Lett. 589, 1257-1265 (2015) disclose that MSC-derived exosomes may improve in vitro myogenesis in C2C12 myoblasts and angiogenesis in HUVECs, while accelerating in vivo skeletal muscle regeneration in a cardiotoxin-induced muscle injury model. This study suggests that the MSC-derived exosomes promote muscle regeneration by enhancing myogenesis and angiogenesis, which is at least in part mediated by miRNAs such as miR-494

In addition, Bier A, Berenstein P, Kronfeld N, et al., 2018. Placenta-derived mesenchymal stromal cells and their exosomes exert therapeutic effects in Duchenne muscular dystrophy. Biomaterials, 174:67-78 found that the exosomes isolated from placenta-derived MSCs (PL-MSCs) promote the differentiation of myoblasts from Duchenne patients and mdx mice and decrease the expression of fibrogenic genes in myoblasts of patients with DMD.

Yan B, Zhang Y, Liang C, Liu B, Ding F, Wang Y, Zhu B, Zhao R, Yu X Y, Li Y. Stem cell-derived exosomes prevent pyroptosis and repair ischemic muscle injury through a novel exosome/circHIPK3/FOXO3a pathway. Theranostics. 2020 May 18; 10(15):6728-6742 showed that human umbilical cord MSC-Exosome treatment significantly promotes running distance and muscle force in a hindlimb ischemia mice model.

Iyer S R, Scheiber A L, Yarowsky P, Henn R F, Otsuru S, Lovering R M (Exosomes Isolated From Platelet-Rich Plasma and Mesenchymal Stem Cells Promote Recovery of Function After Muscle Injury. The American Journal of Sports Medicine. 2020; 48(9):2277-2286)) revealed that muscles treated with exosomes of bone marrow-derived MSC (BMSC-Exos) facilitate myogenesis after muscle injury.

Wang C, Song W, Chen B, Liu X, He Y (Exosomes Isolated From Adipose-Derived Stem Cells: A New Cell-Free Approach to Prevent the Muscle Degeneration Associated With Torn Rotator Cuffs. The American Journal of Sports Medicine. 2019; 47(13):3247-3255) demonstrated that human adipose stem cells-derived exosomes (ASCs-Exos) significantly improve the regeneration and biomechanical properties of torn rotator cuff muscles by ameliorating atrophy and degeneration.

Yan et al. (2020) showed that hucMSC-Exo treatment significantly promotes running distance and muscle force in a hindlimb ischemia mice model.

The studies cited above indicate that MSCs-derived and placenta-derived exosomes exert beneficial roles in myogenesis. Hence, human placenta derived exosomes could modulate the loss of muscle while creating no negative impact to the intended effect of weight loss by the GLP-1 drugs. Human placenta derived exosomes, based on the scientific literature, could provide an added value and benefit in the older population who may experience additional loss of muscle mass for weight loss treatment with GLP-1 drugs.

### II. Application of PDA-001 Cells for Modulating Muscle Loss In Vivo

### Background

T critical limb ischemia model can also be used to demonstrate that PDA-001 cells modulate muscle cell loss, and thus readily have applications in addressing skeletal muscle loss that occurs in subjects being treated with a GLP-1 agonist. In particular, this mice model of ischemic damage is used here to demonstrate efficacy of placenta derived biological material having a continuous lipid bilayer, e.g., PDA-001 cells, in modulating muscle loss in a subject being treated with a GLP-1 agonist.

Materials and Methods
Cells

| | |
|---|---|
| Name: | PDA-001 |
| Storage conditions: | −19° C. to −194° C. |

Vehicle: set forth in Table 1 below.

TABLE 1

| | | | Lot | Expiration |
|---|---|---|---|---|
| Component | Producer | Catalog/NDC Number | Number | Date |
| Dextran | Hospira | NDC 0409-7419-03 | 83-185-JT | 1 Nov. 2011 |
| Hypothermosol | BioLife Solutions | 101104 | 10021 | 1 May 2011 |
| HSA | OctaPharma | NDC 67467-643-01 | A921E6671 | 23 May 2012 |
| DMSO | Bioniche Pharma | Cryoserv (no cat. | 100413 | 1 Mar. 2013 |
| | USA LLC | number) | | |

| | |
|---|---|
| Name: | PDA Cryo (55% Dextran, 5% DMSO, 40% HSA) |
| Storage conditions: | −80° C. |
| Name: | Hypothermosol vehicle (57.5% Dextran, 2.5% |
| | DMSO, 40% HSA) |
| Storage conditions: | −80° C. |

Positive Control

| | |
|---|---|
| Name: | VEGF |
| Manufacturer: | Sigma |
| Lot number: | 080M1700 |
| Batch supplied by: | PHARMASEED |
| Storage conditions: | −20° C. |

### Formulation

All test items and vehicle control were supplied by the sponsor as ready-to-use solutions. PDA-001 cells were delivered to PHARMASEED at the beginning of the study and were stored as depicted above

Experimental Model

| | |
|---|---|
| Animal Model Selection: | Hind limb ischemia |
| Species/Strain: | Balb/c Mice |
| Gener/Number/Age | Male/206/9 weeks |
| Source: | Harlan Laboratories, Inc. |
| Body Fat: | The average body weight was 24.6 g at study |
| | initiation (Day 0). The minimum and maximum |
| | weights of the group were within a range |
| | of ±20% of group mean weight. |
| Acclimation Period: | 11-13 days |
| Identification: | 3 position ear notching and cage cards |

### Animal Management

### Housing

Animal handling was performed according to the National Institute of Health (NIH) and the Association for Assessment and Accreditation of Laboratory Animal Care (AAALAC).

Animals were housed in polysulfone (PSU) cages (7 to 8 per cage), measuring 42.5 cm×265.6 cm×18.5 cm, with stainless steel top grill having facilities for pelleted food and drinking water in clear glass; polycarbonate bottle; bedding-steam-sterilized clean paddy husk (Harlan, Sanichip, cat. number 106S8216) was used and bedding material was changed along with the cage at least twice a week.

### Diet

Animals were fed ad libitum a commercial rodent diet (Teklada Certified Global 18% Protein Diet cat #: 106S8216). Animals have free access to autoclaved and acidified drinking water (pH between 2.5 and 3.0) obtained from the municipality supply.

### Contaminants

Reasonably expected contaminants in food and water supplies did not have the potential to influence outcome of this test.

### Environment Conditions

Animals were housed under the standard laboratory conditions, air-conditioned and filtered (HEPA F6/6) with adequate fresh air supply (minimum 15 air changes/hour). Animals were kept in a climate-controlled environment. Temperature range was between 20° C. to 24° C., and RH was between 30% to 70% with 12 hours light, and 12 hours dark cycle.

### Randomization

Animals were allocated into test groups according to the blood flow results 24 hours after hind limb ischemia initiation.

### Facility

PHARMASEED conducts its preclinical studies in 736-square-meter facility, in Ness-Ziona, Israel. The PHARMASEED facility houses a 95-square-meter vivarium, with a surgery suite and several procedure rooms.

### Veterinary Care

Animals were inspected on arrival and found to be healthy.

### Ethical Committee

This study was performed after approval by “The Israel Board for Animal Experiments,” and in compliance with “The Israel Animal Welfare Act,” approval number IL-11-01-004.

### Experimental Design and Conditions

### Humane Endpoints

No animals were found in a moribund condition or showed severe pain and enduring signs of severe distress.

### Study Initiation Definition

Surgery Day is defined as “Day 1” in this Study.

### Preparation and Anesthesia

On the day of surgery, anesthesia was induced by 1.5% to 3.0% isoflurane, 1.5% N2O, and 0.5% O₂.

### Surgical Procedure

Under anesthesia, the mouse was placed ventral side up. A 0.5 to 1.0 cm incision was made in the skin in the inguinal area. The femoral artery was ligated twice with 6-0 silk thread and transected between the ligatures. The wound was closed with 4-0 silk thread, and the mouse was allowed to recover.

### Cell Implantation

On days 2-3, 24, or 48-hours post-surgery, each animal was injected intramuscularly at 2 sites: the proximal and the distal side of the surgical wound, or intravenous in the tail vein with a composition comprising about 1×10⁵ to 1×10⁶ PDA-001 cells for intramuscular injection. The animals were injected 25 μL in each site for a total of 50 μL per animal. For intravenous injection, the animals were injected 50 μL per animal (see Table 5). Each treated group was divided into 2 subgroups. The first subgroup was treated 24 hours after surgery, and the second group was treated 48 hours after surgery.

TABLE 2
Group Allocation

| | | Cell | Route of |
|---|---|---|---|
| Group | Treatment (Lot) | Amount | Administration |
| A (40, 42, 55, 68, 86, 90, 97, 105, | Negative control | 0 | IM into 2 sites |
| 109, 130, 134, 135, 148, 155, | (dextran) | | |
| 175, 183, and 190) | | | |
| B (52, 56, 67, 71, 78, 84, 112, | Positive control | 0 | IM into 2 sites |
| 116, 123, 127, 156, 164, 191, | VEGF | | |
| 192, 194, 195, 197, 200, and 205) | | | |
| C (38, 51, 57, 62, 72, 77, 93, 94, | PDA-001 Local D1 | 1 × 10⁵ | IM into 2 sites |
| 108, 129, 146, 152, 168, 172, | | | |
| 180, 188, 196, 203, 209, and 213) | | | |
| D (31, 32, 35, 37, 45, 59, 102, 104, | PDA-001 Local D2 | 3 × 10⁵ | IM into 2 sites |
| 107, 122, 140, 149, 150, 157, | | | |
| 158, and 171) | | | |
| E (46, 61, 74, 81, 83, 85, 95, 110, | PDA-001 Local D3 | 1 × 10⁶ | IM into 2 sites |
| 121, 125, 143, 154, 161, 177, | | | |
| 181, 184, 198, 207, and 210) | | | |
| F (36, 49, 60, 63, 66, 69, 88, 96, | PDA-001 IV D3 | 1 × 10⁶ | IV in tail vein |
| 115, 117, 131, 138, 145, 153, | | | |
| 182, and 204) | | | |
| L (1, 3, 5, 11, 12, 13, 14, 15, 16, | Negative control | 0 | IM into 2 sites |
| 19, 20, 21, 23, 24, 29, 206, and | Hypothermasol | | |
| 208) | | | |

### Tests and Evaluation

### Body Weight

Body weight was measured on study day prior to the surgery and once weekly thereafter.

### Blood Flow Measurement

Blood flow in legs from both sides were measured with a noncontact laser Doppler before surgery and on Days 1, 7, 15, 21, 28, 35, 42, and 49 post operation. Blood flow measurements were expressed as the ratio of the flow in the ischemic limb to that in the normal limb.

### Macroscopic Evaluation of Ischemic Severity

Macroscopic evaluation of the ischemic limb was done once a week post operation by using morphological grades for necrotic area.

TABLE 3
Morphological Grades for Necrotic Area

| Grade | Description |
|---|---|
| 0 | absence of necrosis |
| 1 | necrosis limiting to toes (toe loss) |
| 2 | necrosis extending to a dorsum pedis (foot loss) |
| 3 | necrosis extending to a crus (knee loss) |
| 4 | necrosis extending to a thigh (total hind limb loss) |

### In Vivo Assessment of Limb Function and Ischemic Damage

Semiquantitative assessment of impaired use of the ischemic limb was performed once a week post-surgery using the following scale.

TABLE 4
Assessments of Limb Function

| Grade | Description |
|---|---|
| 0 | flexing the toes to resist gentle traction of the tail |
| 1 | plantar flexion |
| 2 | no dragging but no plantar flexion |
| 3 | dragging of foot |

Limb function was graded as “Not applicable” in cases of partial or full limb amputation. In such cases, blood flow measurements were not included in the statistical analysis.

### Ultrasound Image

At study termination on Day 50, an ultrasound image analysis of the injured leg was done.

### Mice Preparation

Mice were anesthetized with 2% inhalation anesthesia (isoflurane, Halocarbon Products, River Edge, NJ) delivered with oxygen, using a nonrebreathing anesthetic delivery system (Summit Anastasia Solutions, Bend, OR). Anesthetized mice were placed on a heating pad to maintain body temperature and to minimize temperature-induced changes in blood flow. Hair surrounding the imaged area was removed before imaging. An imaging gel was placed on the limb, and a 27-gauge needle was inserted into the tail vein for repeated intravenous injections.

### Ultrasound Imaging

To retain the physiologic conditions and to reduce the volume injected into the mice, first-pass ultrasound imaging was performed. Bolus contrast media (Definity Perflutren Lipid Microsphere, Bristol-Myers Squibb Medical Imaging, Inc, N. Billerica, MA) was injected through the tail vein in a volume of 200 μL (20 mg/mouse and low-intensity and nondestructive CM-enhanced ultrasound imaging was performed on the first time that the CM was washed into the limb). Ultrasound measurements were carried out at 40 frames/second, with a fixed transducer (Vevo2100, VisualSonic, CA). Contrast media (CM) enhancement was measured for 15 seconds at a rate of 40 frames/second (starting before the injection), until CM enhancement reached plateau. Imaging settings were standardized and unchanged throughout the experiment. No major near-field artifacts were encountered. The use of CM enables the detection of smaller blood vessels relative to large blood vessels observed by Doppler ultrasound.

### Image Analysis

Linear contrast agent imaging refers to visualizing the micro bubbles CM using B-Mode imaging (anatomy mode) and then overlaying the contrast specific signal on this image. This was done by applying a reference subtraction algorithm to apply a green contrast overlay on the image to aid in the visualization and quantification of the contrast specific signal. A mouse cursor was used to outline the limb regions of interest (ROI) on the B-mode ultrasound image based on structural criteria and echogenicity parameters. CM enhancement was calculated and a perfusion curve plotted from time series images (clips) using the Vevo2100 analysis tool. This curve showed the flow in the ROI. The slope of the curve (parameter B) indicated relative blood velocity, while the plateau (parameter A) indicated relative blood volume.

### Tissue Fixation and Gross Pathology

After ultrasound analysis gross pathology was performed. The quadriceps muscles of the ischemic and control legs were removed, weighted, fixed in HOPE fixative, imbedded in paraffin, and sent to sponsor. The lymph nodes and spleen were removed, snap-frozen and sent to sponsor.

### Results

In this study, a stable severe ischemia model was applied to assess whether placet derived adherent cells, such as PDA-001 cells, can modulate muscle loss in a stable severe ischemia model. If so, such cells readily have applications in modulating muscle loss in a subject being treated with a GLP-1 agonist.

### Mortality and Clinical Signs

Five mice died during the study. Among them, mouse number 2 from Group K, mice numbers 74 and 121 from Group E, mouse number 108 from Group C, and mouse number 43 from Group I. No clinical signs were observed in all animal groups.

### Body Weights

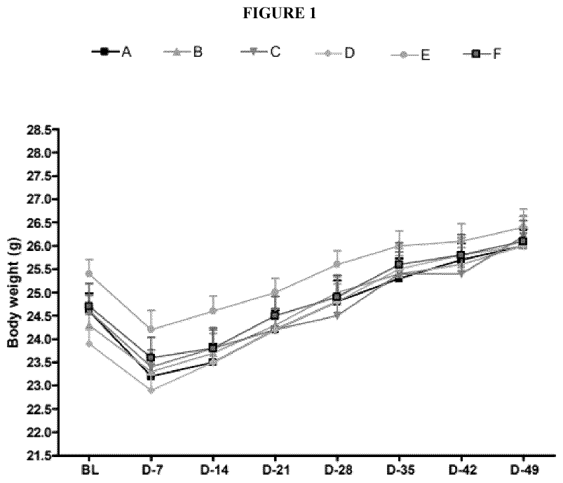

Throughout the study, no statistically significant differences in body weight were observed among all treated groups of animals (FIG. 1).

### Blood Flow Measurement

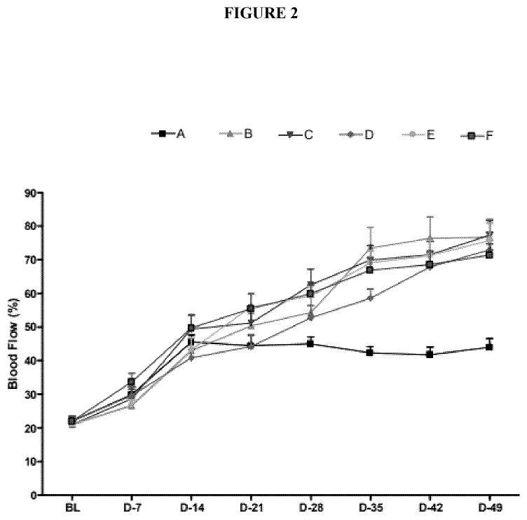

Throughout the study, statistically significant improvement in blood flow was observed in all animal groups treated with PDA-001 cells as compared to dextran-treated control group. This improvement was found from Day 21 after hind limb ischemia initiation and increased up to Day 49 postischemia (FIG. 2).

### Two Way ANOVA Repeated Measurements, Bonferroni Post Hoc Tests—PDA-001

Comparison of control Group A to positive control VEGF Group B showed that there was a statistically significant difference starting at Day 35 up to Day 49 (p<0.001). Comparison of control group to PDA001 groups showed that there was a statistically significant difference at Day 28 up to Day 49 (p<0.01-0.001) for Groups C, E, and F. For Group D, statistically significant difference was found at Day 35 up to Day 49 (p<0.01). Two way ANOVA results for PDA-001 are set forth in Table 5.

TABLE 5
Two-way ANOVA Results - PDA-001

| Two-way ANOVA | % of total | |
|---|---|---|
| Source of Variation | variation | P value |
| Interaction | 6.08 | P < 0.0001 |
| Column Factor | 5.45 | P < 0.0001 |
| corr BF | 55.15 | P < 0.0001 |

| | P value | |
|---|---|---|
| Source of Variation | summary | Significant? |
| Interaction | *** | Yes |
| Column Factor | *** | Yes |
| corr BF | *** | Yes |

| | | Sum-of- | | |
|---|---|---|---|---|
| Source of Variation | Df | squares | Mean square | F |
| Interaction | 35 | 20340 | 581.3 | 3.392 |
| Column Factor | 5 | 18240 | 3648 | 21.29 |
| corr BF | 7 | 184400 | 26350 | 153.8 |
| Residual | 650 | 111400 | 171.4 | |
| Number of missing | 118 | | | |
| values | | | | |

| Bonferroni posttests | | | | |
|---|---|---|---|---|
| A vs. B | | | | |
| corr BF | A | B | Difference | 95% CI of diff. |
| BL | 22 | 21 | −1 | −18.60 to 16.60 |
| D-7 | 29.9 | 26.6 | −3.3 | −20.64 to 14.04 |
| D-14 | 45.5 | 43 | −2.5 | −19.84 to 14.84 |
| D-21 | 44.5 | 50.4 | 5.9 | −11.44 to 23.24 |
| D-28 | 45 | 54.3 | 9.3 | −8.040 to 26.64 |
| D-35 | 42.3 | 73.5 | 31.2 | 13.86 to 48.54 |
| D-42 | 41.8 | 76.4 | 34.6 | 17.26 to 51.94 |
| D-49 | 44 | 76.7 | 32.7 | 15.36 to 50.04 |

| corr BF | Difference | t | P value | Summary |
|---|---|---|---|---|
| BL | −1 | 0.2016 | P > 0.05 | ns |
| D-7 | −3.3 | 0.6751 | P > 0.05 | ns |
| D-14 | −2.5 | 0.5115 | P > 0.05 | ns |
| D-21 | 5.9 | 1.207 | P > 0.05 | ns |
| D-28 | 9.3 | 1.903 | P > 0.05 | ns |
| D-35 | 31.2 | 6.383 | P < 0.0001 | *** |
| D-42 | 34.6 | 7.079 | P < 0.0001 | *** |
| D-49 | 32.7 | 6.69 | P < 0.0001 | *** |

| A vs. C | | | | |
|---|---|---|---|---|
| corr BF | A | C | Difference | 95% CI of diff. |
| BL | 22 | 21 | −1 | −17.96 to 15.96 |
| D-7 | 29.9 | 28.7 | −1.2 | −17.89 to 15.49 |
| D-14 | 45.5 | 49.4 | 3.9 | −13.44 to 21.24 |
| D-21 | 44.5 | 51.2 | 6.7 | −10.64 to 24.04 |
| D-28 | 45 | 62.5 | 17.5 | 0.1596 to 34.84 |
| D-35 | 42.3 | 69.9 | 27.6 | 10.26 to 44.94 |
| D-42 | 41.8 | 71.5 | 29.7 | 12.36 to 47.04 |
| D-49 | 44 | 77.3 | 33.3 | 15.96 to 50.64 |

| corr BF | Difference | t | P value | Summary |
|---|---|---|---|---|
| BL | −1 | 0.2092 | P > 0.05 | ns |
| D-7 | −1.2 | 0.2551 | P > 0.05 | ns |
| D-14 | 3.9 | 0.7979 | P > 0.05 | ns |
| D-21 | 6.7 | 1.371 | P > 0.05 | ns |
| D-28 | 17.5 | 3.58 | P < 0.01 | ** |
| D-35 | 27.6 | 5.647 | P < 0.001 | *** |
| D-42 | 29.7 | 6.076 | P < 0.001 | *** |
| D-49 | 33.3 | 6.813 | P < 0.001 | *** |

| A vs. D | | | | |
|---|---|---|---|---|
| corr BF | A | D | Difference | 95% CI of diff. |
| BL | 22 | 22 | 0 | −16.96 to 16.96 |
| D-7 | 29.9 | 29.6 | −0.3 | −16.99 to 16.39 |
| D-14 | 45.5 | 40.8 | −4.7 | −21.39 to 11.99 |
| D-21 | 44.5 | 44.2 | −0.3 | −16.99 to 16.39 |
| D-28 | 45 | 52.7 | 7.7 | −8.990 to 24.39 |
| D-35 | 42.3 | 58.6 | 16.3 | −0.3904 to 32.99 |
| D-42 | 41.8 | 67.8 | 26 | 9.310 to 42.69 |
| D-49 | 44 | 72.9 | 28.9 | 12.21 to 45.59 |

| corr BF | Difference | t | P value | Summary |
|---|---|---|---|---|
| BL | 0 | 0 | P > 0.05 | ns |
| D-7 | −0.3 | 0.06377 | P > 0.05 | ns |
| D-14 | −4.7 | 0.999 | P > 0.05 | ns |
| D-21 | −0.3 | 0.06377 | P > 0.05 | ns |
| D-28 | 7.7 | 1.637 | P > 0.05 | ns |
| D-35 | 16.3 | 3.465 | P < 0.01 | ** |
| D-42 | 26 | 5.526 | P < 0.001 | *** |
| D-49 | 28.9 | 6.143 | P < 0.001 | *** |

| A vs. E | | | | |
|---|---|---|---|---|
| corr BF | A | E | Difference | 95% CI of diff. |
| BL | 22 | 21 | −1 | −17.96 to 15.96 |
| D-7 | 29.9 | 26.9 | −3 | −19.18 to 13.18 |
| D-14 | 45.5 | 43.3 | −2.2 | −18.62 to 14.22 |
| D-21 | 44.5 | 56 | 11.5 | −5.190 to 28.19 |
| D-28 | 45 | 59.2 | 14.2 | −2.795 to 31.20 |
| D-35 | 42.3 | 69.1 | 26.8 | 9.805 to 43.80 |
| D-42 | 41.8 | 71.3 | 29.5 | 12.50 to 46.50 |
| D-49 | 44 | 75.8 | 31.8 | 14.80 to 48.80 |

### Two-Way ANOVA Repeated Measurements, Bonferroni Post Hoc Tests (“Last Measure Carried Forward” Method)—PDA-001

Comparison of control Group A to positive control VEGF Group B showed that there was a statistically significant difference starting at Day 35 up to Day 49 (p<0.05). Comparison of control group to PDA-001 groups showed that there was a statistically significant difference at Day 35 up to Day 49 (p<0.01-0.001) for groups D, E, and F. For Group C, statistically significant differences were found at Day 42 and 49 (p<0.05).

TABLE 6
Two-way ANOVA Results (“Last Measure Carried
Forward” method) - PDA-001

| Two-way ANOVA | % of total | |
|---|---|---|
| Source of Variation | variation | P value |
| Interaction | 4.07 | 0.0299 |
| Column Factor | 4.75 | P < 0.0001 |
| corr BF last | 41.35 | P < 0.0001 |
| | P value | |
| Source of Variation | summary | Significant? |
| Interaction | * | Yes |
| Column Factor | *** | Yes |
| corr BF last | *** | Yes |

| | | Sum-of- | | |
|---|---|---|---|---|
| Source of Variation | Df | squares | Mean square | F |
| Interaction | 35 | 13560 | 387.4 | 1.518 |
| Column Factor | 5 | 15810 | 3162 | 12.39 |
| corr BF last | 7 | 137700 | 19670 | 77.07 |
| Residual | 650 | 165900 | 255.2 | |
| Number of missing | 118 | | | |
| values | | | | |

| Bonferroni posttests | | | | |
|---|---|---|---|---|
| A vs. B | | | | |
| corr BF last | A | B | Difference | 95% CI of diff. |
| BL | 20.9 | 21.1 | 0.2 | −21.27 to 21.67 |
| D-7 | 29.9 | 24.6 | −5.3 | −26.46 to 15.86 |
| D-14 | 44.6 | 37.6 | −7 | −28.16 to 14.16 |
| D-21 | 43.6 | 42.3 | −1.3 | −22.46 to 19.86 |
| D-28 | 44.2 | 45.2 | 1 | −20.16 to 22.16 |
| D-35 | 41.6 | 59.3 | 17.7 | −3.460 to 38.86 |
| D-42 | 41.2 | 60.1 | 18.9 | −2.260 to 40.06 |
| D-49 | 43.2 | 60.3 | 17.1 | −4.060 to 38.26 |

| corr BF last | Difference | t | P value | Summary |
|---|---|---|---|---|
| BL | 0.2 | 0.03304 | P > 0.05 | ns |
| D-7 | −5.3 | 0.8886 | P > 0.05 | ns |
| D-14 | −7 | 1.174 | P > 0.05 | ns |
| D-21 | −1.3 | 0.218 | P > 0.05 | ns |
| D-28 | 1 | 0.1677 | P > 0.05 | ns |
| D-35 | 17.7 | 2.967 | P < 0.05 | * |
| D-42 | 18.9 | 3.169 | P < 0.05 | * |
| D-49 | 17.1 | 2.867 | P < 0.05 | * |

| A vs. C | | | | |
|---|---|---|---|---|
| corr BF last | A | C | Difference | 95% CI of diff. |
| BL | 20.9 | 21.2 | 0.3 | −20.39 to 20.99 |
| D-7 | 29.9 | 27 | −2.9 | −23.27 to 17.47 |
| D-14 | 44.6 | 41.8 | −2.8 | −23.96 to 18.36 |
| D-21 | 43.6 | 43.2 | −0.4 | −21.56 to 20.76 |
| D-28 | 44.2 | 51.7 | 7.5 | −13.66 to 28.66 |
| D-35 | 41.6 | 57.2 | 15.6 | −5.560 to 36.76 |
| D-42 | 41.2 | 57.8 | 16.6 | −4.560 to 37.76 |
| D-49 | 43.2 | 60.9 | 17.7 | −3.460 to 38.86 |

| corr BF last | Difference | t | P value | Summary |
|---|---|---|---|---|
| BL | 0.3 | 0.05143 | P > 0.05 | ns |
| D-7 | −2.9 | 0.5051 | P > 0.05 | ns |
| D-14 | −2.8 | 0.4694 | P > 0.05 | ns |
| D-21 | −0.4 | 0.06706 | P > 0.05 | ns |
| D-28 | 7.5 | 1.257 | P > 0.05 | ns |
| D-35 | 15.6 | 2.615 | P > 0.05 | ns |
| D-42 | 16.6 | 2.783 | P < 0.05 | * |
| D-49 | 17.7 | 2.967 | P < 0.05 | * |

| A vs. D | | | | |
|---|---|---|---|---|
| corr BF last | A | D | Difference | 95% CI of diff. |
| BL | 20.9 | 21.6 | 0.7 | −19.99 to 21.39 |
| D-7 | 29.9 | 29.6 | −0.3 | −20.67 to 20.07 |
| D-14 | 44.6 | 40.8 | −3.8 | −24.17 to 16.57 |
| D-21 | 43.6 | 44.2 | 0.6 | −19.77 to 20.97 |
| D-28 | 44.2 | 52.7 | 8.5 | −11.87 to 28.87 |
| D-35 | 41.6 | 58.6 | 17 | −3.367 to 37.37 |
| D-42 | 41.2 | 67.8 | 26.6 | 6.233 to 46.97 |
| D-49 | 43.2 | 72.1 | 28.9 | 8.533 to 49.27 |

| corr BF last | Difference | t | P value | Summary |
|---|---|---|---|---|
| BL | 0.7 | 0.12 | P > 0.05 | ns |
| D-7 | −0.3 | 0.05226 | P > 0.05 | ns |
| D-14 | −3.8 | 0.6619 | P > 0.05 | ns |
| D-21 | 0.6 | 0.1045 | P > 0.05 | ns |
| D-28 | 8.5 | 1.481 | P > 0.05 | ns |
| D-35 | 17 | 2.961 | P < 0.05 | * |
| D-42 | 26.6 | 4.633 | P < 0.001 | *** |
| D-49 | 28.9 | 5.034 | P < 0.001 | *** |

| A vs. E | | | | |
|---|---|---|---|---|
| corr BF last | A | E | Difference | 95% CI of diff. |
| BL | 20.9 | 20.9 | 0 | −20.69 to 20.69 |
| D-7 | 29.9 | 26.9 | −3 | −22.74 to 16.74 |
| D-14 | 44.6 | 42.3 | −2.3 | −22.34 to 17.74 |
| D-21 | 43.6 | 54.3 | 10.7 | −9.667 to 31.07 |
| D-28 | 44.2 | 56.8 | 12.6 | −8.139 to 33.34 |
| D-35 | 41.6 | 64.3 | 22.7 | 1.961 to 43.44 |
| D-42 | 41.2 | 66.1 | 24.9 | 4.161 to 45.64 |
| D-49 | 43.2 | 69.8 | 26.6 | 5.861 to 47.34 |

| corr BF last | Difference | t | P value | Summary |
|---|---|---|---|---|
| BL | 0 | 0 | P > 0.05 | ns |
| D-7 | −3 | 0.5392 | P > 0.05 | ns |
| D-14 | −2.3 | 0.4072 | P > 0.05 | ns |
| D-21 | 10.7 | 1.864 | P > 0.05 | ns |
| D-28 | 12.6 | 2.158 | P > 0.05 | ns |
| D-35 | 22.7 | 3.883 | P < 0.001 | *** |
| D-42 | 24.9 | 4.259 | P < 0.001 | *** |
| D-49 | 26.6 | 4.55 | P < 0.001 | *** |

| A vs. F | | | | |
|---|---|---|---|---|
| corr BF last | A | F | Difference | 95% CI of diff. |
| BL | 20.9 | 22.2 | 1.3 | −19.39 to 21.99 |
| D-7 | 29.9 | 33.7 | 3.8 | −16.57 to 24.17 |
| D-14 | 44.6 | 49.7 | 5.1 | −15.27 to 25.47 |
| D-21 | 43.6 | 55.5 | 11.9 | −8.467 to 32.27 |
| D-28 | 44.2 | 59.9 | 15.7 | −4.667 to 36.07 |
| D-35 | 41.6 | 66.9 | 25.3 | 4.933 to 45.67 |
| D-42 | 41.2 | 68.6 | 27.4 | 7.033 to 47.77 |
| D-49 | 43.2 | 71.4 | 28.2 | 7.833 to 48.57 |

| corr BF last | Difference | t | P value | Summary |
|---|---|---|---|---|
| BL | 1.3 | 0.2229 | P > 0.05 | ns |
| D-7 | 3.8 | 0.6619 | P > 0.05 | ns |
| D-14 | 5.1 | 0.8883 | P > 0.05 | ns |
| D-21 | 11.9 | 2.073 | P > 0.05 | ns |
| D-28 | 15.7 | 2.735 | P > 0.05 | ns |
| D-35 | 25.3 | 4.407 | P < 0.001 | *** |
| D-42 | 27.4 | 4.773 | P < 0.001 | *** |
| D-49 | 28.2 | 4.912 | P < 0.001 | *** |

### Ultrasound Imaging

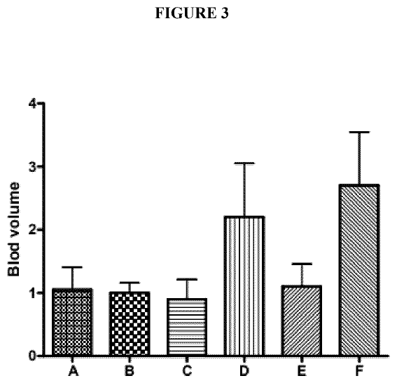

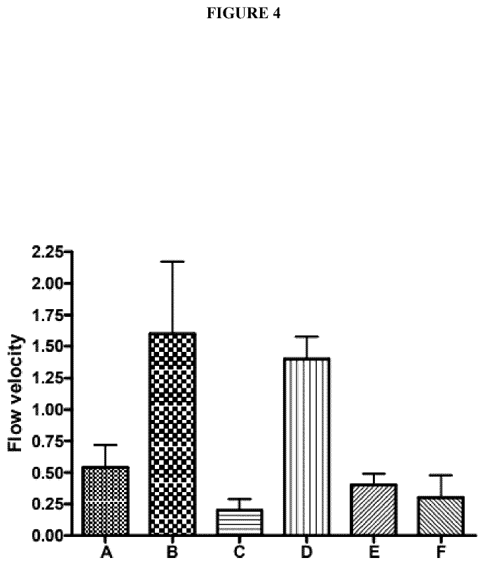

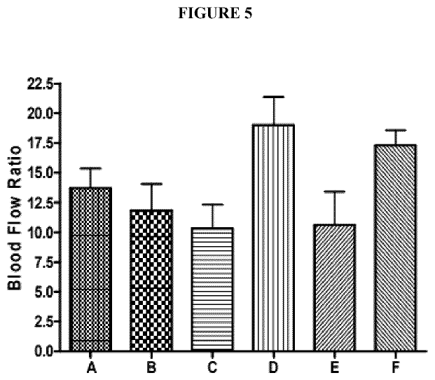

Contrast-enhanced ultrasound (CEU) imaging of the proximal hind limb adductor muscles was performed during the IV bolus of contrast microbubbles. The technique characterized large intramuscular vessels remodeling in different vascular compartments during ischemia-mediated angiogenesis. CEU data were analyzed to measure capillary perfusion and functional noncapillary microvascular blood volume. Changes in perfusion were temporally related to expansion of noncapillary microvascular blood volume on CEU, which was associated with an arteriogenic response. Seven weeks after iliac artery ligation, blood flow volume in ischemic muscles was reduced in control animals and markedly increased in PDA001-treated Groups D and F (FIG. 3). Similar improvement was found in blood flow velocity FIG. 4) and blood flow ratio (FIG. 5).

### In Vivo Assessment of Ischemic Severity

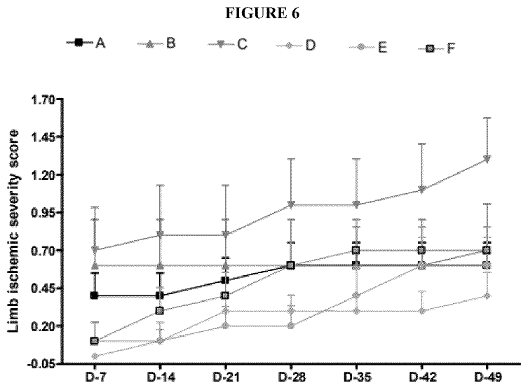

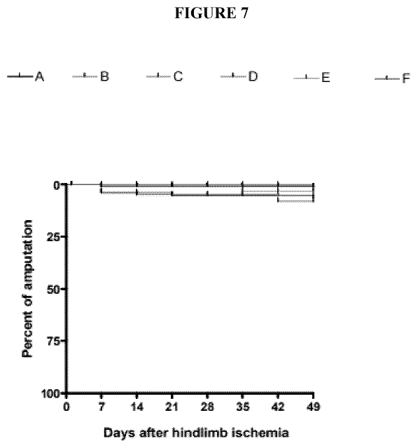

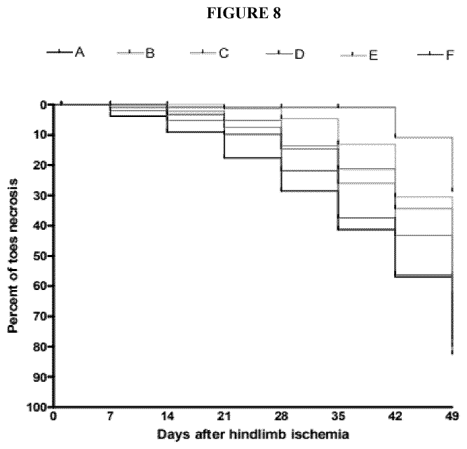

The ischemic limb was macroscopically evaluated on Days 7 up to 49 by using graded morphological scales for necrotic area. In all animal groups treated with PDA cells, except for animals of Group D (local cell administration in concentration of 3×10⁵), limb amputation was found. Percent of amputation in control Group A was 6%, in Group B-26%, in Group C-25%, in Groups E and F-6% (FIG. 6). No amputation was found in Groups J and K (FIG. 7). No significant differences were found in toes necrosis dynamics. Only in Groups B (VEGF positive control) and D (PDA001 cells 1×10⁴), do the number of animals with toes necrosis decreased as compared to control Group A (FIG. 8). 0% represents no toe necrosis; the decrease reflects the increase of necrosis through the study.

### In Vivo Assessment of Limb Function

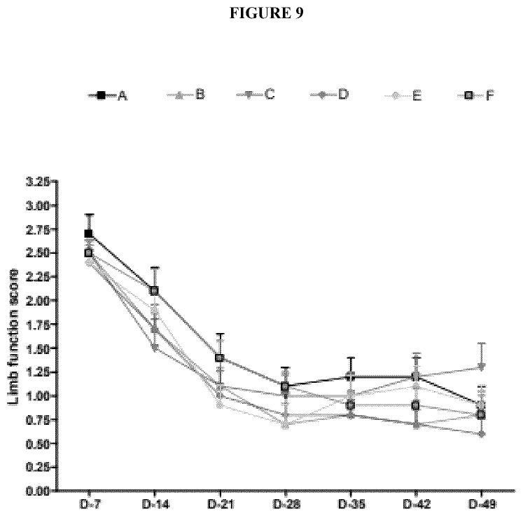

Semiquantitative assessment of impaired use of the ischemic limb was performed on Days 7 up to 49 by using graded functional scales. An improvement in limb function was found in treated Groups B, D, and E versus Group A (negative control, dextran), and in Groups I and J versus Group L (negative control, hypothermasol); however, the differences were not statistically significant (FIG. 9).

### Discussion

Impaired angiogenesis is one of the features of ischemic diseases. The most established target for therapeutic angiogenesis has been VEGF and its receptors. However, clinical trials to alleviate ischemia were disappointing, indicating the need for the new molecules and therapeutic targets to treat ischemic diseases. Stem cell therapy is a promising approach in cardiovascular medicine. In order to assess the therapeutic activity of PDA-001 stem cells in ischemic tissue, a mouse hind limb ischemia model was used. It was found that a single local or IV administration of PDA-001 cells to the ischemic limb restored blood perfusion to 60% to 70% of its normal values, respectively. Blood flow perfusion restoration was compatible with other results that those cells caused, similar improvement in limb function, and decreased ischemic severity in mice hind limb ischemia model. No dose dependent effect of implanted cells was for PDA-001 cells. In this study, treatment of the ischemic muscle with PDA-001 resulted in a greater and more rapid recovery of blood flow of capillary micro vessels, as measured by surface laser-Doppler imaging. Contrast-enhanced ultrasound imaging provides a noninvasive and precise method to assess the response to occlusive peripheral arterial disease, as well as the therapeutic response to stem cells treatment. This method detects blood perfusion in small penetrating arteries and medium-sized arterioles in the ischemic proximal hind limb adductor muscle. It was found that capillary perfusion and functional noncapillary microvascular blood volume increased in animals treated with PDA-001 cells. Although this improvement did not reach statistically significant level (except Group J), partly due to the small group size, the trend of improvement was clear.

### Conclusions

PDA-001 cell implantation in mouse model of chronic hind limb ischemia improved blood perfusion and limb functional recovery and resulted in changes in microvascular limb muscles vessel morphology and flow, resulting in improved muscle strength. Hence, these results demonstrate that placenta derived adherent cells, such as PDA-001 and PDA-002, readily have applications in modulating muscle loss in a subject being treated with a GLP-1 agonist.

### II. Use of PDA-002 Cells to Ameliorate Muscle Loss and Increase Muscle Strength

This study was designed to evaluate the therapeutic activities PDA-002 cells in a rat hind limb ischemia model. The level of ischemia was determined by serial measurements using an observational rating scale, and by serial blood flow measurements (relative to the intact opposite limb) made by laser Doppler scanning. It has been reported that skeletal muscle mass decreases in patients suffering from chronic limb threatening ischemia (Ferreira et al., Annals of Vascular Surgery (88:164-173 (2023)). Hence, demonstrating in this model that intramuscular administration of PDA-002 cells increase muscle strength and modulate muscle loss demonstrates that such cells have applications in ameliorating muscle loss in subjects being treated with a GLP-1 agonist.

### Methods

### 1. Animals, Housing and Diet

Fifty-six male Sprague-Dawley rats, 250 g to 275 g (59 to 62 days old) were ordered 7 to 10 days prior to surgery (Charles River Laboratories, 8 extra animals included). They were allowed free access to food and water. Animals were assigned sequential identification numbers using permanent marker on the tail. The animals were observed the day prior to surgery, and those appearing to be in poor health were excluded from the study. Animals were housed in rooms provided with filtered air at a temperature of 21±2° C. and 50%±20% relative humidity. The room was on an automatic timer for a light/dark cycle of 12 hours on, and 12 hours off with no twilight. SHEPERD'S ¼″ premium corn cob was used for bedding, and 1 NYLABONE (3.5″, Dura bones Petite) and/or a RATRUNNEL (BioServ K3326) was put in each cage. Animals were fed with LAB DIET 5001 chow. Water was provided ad libitum.

The animals were housed 2 per cage before and after surgery, unless severe aggression or injury was displayed, or death of cage mate(s).

### 2. Animal Preparation

Forty-eight, adult, male Sprague-Dawley rats as described above were used for the study. All rats were housed for at least 7 days prior to surgery for acclimation purposes. Rats were randomized and assigned to different groups.

### 3. Surgical Preparation

### (a) Hind Limb Ischemia Model

A modified model of hind limb ischemia (HLI) was used (Couffinhal, 1998; Yu, 2006). After anesthesia, the left femoral artery was gently exposed from the level of the inguinal ligament to the proximal part of the great sapphenous artery under visualization by the operator using a dissecting microscope. The accompanying femoral nerve and femoral vein were dissected free from the artery. The proximal end of the femoral artery, bifurcation of the popliteal artery, and distal portion of the sapphenous artery were ligated by 7-0 nylon suture. All other branches were ligated, and arteriectomy was performed. The incision was closed by a 4-0 or 5-0 suture. Body temperature was maintained at 37.0±1° C. throughout the entire procedure. Buprenorphine (0.05˜0.1 mg/kg; BUPRENEX Reckitt Benckser 107001 Exp 01Mar14) was given subcutaneously (SQ) before the HLI surgery as analgesia.

### (b) Test Article and Vehicle Control

### 1. Test Article

PDA-002 cells (lot number AN01272011) in tan liquid were received from the sponsor in a dry shipper containing liquid nitrogen (gaseous form), at concentrations of approximately 2.0×10⁷ cells/mL. The unit was stored frozen in the dry shipper until use. The pharmaceutical composition comprising PDA-002 cells was thawed and prepared for dose administration as per instructions provided by the sponsor.

### 2. Vehicle Control

The vehicle control contained all the excipients, at the same concentrations as in the PDA-002 composition, but did not contain cells. The lot number was 07142011. The vehicle control was packaged, shipped, and stored as 20 mL frozen aliquots.

### 3. Cell Preparation

Cells and control article were sent in a dry shipper. The cells and control article were stored in the dry shipper until used. A pharmaceutical composition of PDA-002 cells and the control article were thawed each day of injection as per the attached protocol (Appendix 7.4).

### 4. Dosing

PDA-002 cells or control article were administered through tail vein injection (1 mL), or intramuscular injections (50 μL on each side of surgery) on Day 1 following HLI as indicated in Table 1 (Day 0=day of HLI surgery). The solution was slowly injected over 3 minutes (for IV dosing). The injection site was pressed for an additional minute or 2 to limit back flow and bleeding.

TABLE 7
Experimental Groups

| | |
|---|---|
| 1 | IV Treatment |
| | PDA-002 1.00E+04/100 μL., at Day 1 after HLI (N = 6) |
| | (cell conc. = 1.00E+05 cells/mL) |
| 3 | IM Treatment |
| | PDA-002 1.00E+06/100 μL., (50 μL on each side of surgery), |
| | at Day 1 after HLI (N = 6) (cell conc. = 10.00E+06 cells/mL |
| 5 | IM Treatment |
| | PDA-002 3.00E+03/100 μL., (50 μL on each side of surgery), |
| | at Day 1 after HLI (N = 6) (cell conc. = 3.00E+04) |
| 7 | IV Treatment |
| | Vehicle 100 μL, at Day 1 after HLI (N = 6) |
| 8 | IM Treatment |
| | Vehicle 100 μL (50 μL on each side of surgery), at Day 1 after |
| | HLI (N = 6) |

### 5. Randomization and Blinding

Animals were randomly assigned to treatment groups. The code sheet (Table 2) shows assignment of animals. The study schedule (Appendix 7.1) shows the surgery, injection, laser Doppler scanning, and sacrifice time points for each animal. The surgeon and the investigator who performed laser Doppler scanning were blinded to the treatment assignment of each animal.

TABLE 8
Code

| B | D | E | F | H |
|---|---|---|---|---|
| 5 | 7 | 3 | 8 | 2 |
| 11 | 13 | 16 | 14 | 10 |
| 18 | 20 | 24 | 23 | 19 |
| 26 | 31 | 28 | 25 | 29 |
| 33 | 35 | 34 | 38 | 40 |
| 46 | 42 | 44 | 47 | 48 |

Code:
B: IM Treatment, PDA-002 3.00E+03, at Day 1 after PVD
D: IM Treatment, PDA-002 1.00E+06, at Day 1 after PVD
E: IV Treatment, PDA-002 1.00E+04, at Day 1 after PVD
F: IM Treatment, Vehicle, at Day 1 after PVD
H: IV Treatment, Vehicle, at Day 1 after PVD

### 6. Laser Doppler Scanning

Blood flow in the ischemic and contralateral control gastrocnemius muscles were analyzed using a laser Doppler scanning device (PeriScan PIM 3 System, Perimed, Sweden). One day prior to scanning, animals were shaved over the scanned area (the proximal leg). Following shaving, residual hair was removed by the application of NAIR hair remover. Measurements were performed while the animal was anesthetized with isofluorane at Day 1, Day 7, Day 14, Day 21, Day 28, and Day 34. The test animals were secured in a supine position with the hind limbs splayed in a manner whereby each leg was aligned according to midline. The laser Doppler was positioned approximately 20 cm above the surface of the animal for scanning. Upon completion of the scanning, the region of interest (ROI) was defined on the reference (control) leg in a manner to effectively include the gastrocnemius muscle and associated distal vasculature. The ROI was then cloned and positioned over the associated area on the ischemic leg. The size and shape of the ROI was defined on an individual animal basis to best capture the data from animals of different sizes. The “% difference between legs” was defined as ((ROI reference leg−ROI ischemic leg)/ROI reference leg)*100. The “blood flow score” was defined as % difference between legs Day 34−% difference between legs, Day 1. Blood flow scores were then analyzed using one-way ANOVA to determine differences between dose groups.

### 7. Observations—Clinical Scale

Limb ischemia was also evaluated by direct observation using a combined clinical scale that evaluates spontaneous mobility, gait, limb pallor, muscle necrosis, and gangrene. The scale is as follows:
0—Normal
1—Pale foot or abnormal gait
2—Gangrenous tissue<50% of foot without lower limb muscle necrosis
3—Gangrenous tissue<50% of foot with lower limb muscle necrosis
4—Gangrenous tissue of >50% of foot
5—Loss of >50% of lower limb

### 8. Sacrifice (Done at Ekam Imaging)

On Day 35, animals were transported to Ekam for MR imaging. Animals were placed in ventilated shipping containers or cages, with a food/water source, and transported via a private vehicle with inside temperature maintained at 65° F. to 72° F. The approximate travel time to Ekam was 30 minutes. Animal health records accompanied the rats to Ekam, Inc.

Following MR imaging, animals were anesthetized deeply with ketamine/xylazine, and blood (2 to 4 mL) was taken from the heart and snap-frozen, to be processed later for serum. The quadriceps muscles of the ischemic and control legs were removed, weighed, fixed in HOPE fixative, and sent to the sponsor. The draining lymph nodes, including inguinal and popliteal nodes from both sides were removed, placed in separate containers, snap frozen and sent to the sponsor.

### Results

No mortality was found associated with dosing of cells either by IV or IM. There were 3 cases of mortalities associated with the surgery. Animal number 23 died after surgery and was replaced by number 23R. Animal number 36 died after surgery and was replaced by number 36R. Animal number 41 died one day after surgery and was replaced by number 41R.

### 1. Laser Doppler Scanning Results

There were nonsignificant trends of increased blood flow scores in IV PDA-002 1.00E+04, and IM PDA-002. 1.00E+06-treated cell groups compared to appropriate control groups FIG. 1). There were no differences in IM PDA-002 3.00E+03 treated animals versus controls. The full details of the statistical analysis are set forth below:

TABLE 9
ANOVA Table for Laser Doppler Scanning Data
Table 9A: Anova Table for Day 34

| | | Sum of | Mean | F- | P- | | |
|---|---|---|---|---|---|---|---|
| | DF | Squares | Square | Value | Value | Lambda | Power |
| Treatment | 7 | 6024.477 | 860.64 | 1.682 | 0.142 | 11.775 | 0.606 |
| Residual | 39 | 19953.807 | 511.636 | | | | |

Table 9B: Means Table for Day 34
Effect: Treatment

| | Count | Mean | Std. Dev. | Std. Err |
|---|---|---|---|---|
| IM PDA001 3.00E+03 | 6 | 17.743 | 30.494 | 12.449 |
| IM PDA001 1.00E+06 | 6 | 48.102 | 38.726 | 15.81 |
| IV PDA001 1.00E+0.04 | 6 | 43.630 | 19.867 | 8.111 |
| IM Vehicle | 6 | 21.500 | 13.534 | 5.525 |
| IV Vehicle | 6 | 28.522 | 18.537 | 7.568 |

Table 9C: Fisher's PLSD for Day 34
Effect: Treatment
Significance Level: 5%

| | | | |
|---|---|---|---|
| IM PDA001 3.00E+03, IM PDA001 | −30.358 | 26.415 | 0.0254 |
| 1.00E+06 | | | |
| IM PDA001 3.00E+03, IV PDA001 | −25.887 | 26.415 | 0.0545 |
| 1.00E+04 | | | |
| IM PDA001 3.00E+03, IM Vehicle | −3.757 | 26.415 | 0.7751 |
| IM PDA001 3.00E+03, IV Vehicle | −10.778 | 26.415 | 0.4142 |
| IM PDA001 1.00E+06, IV PDA001 | 4.472 | 26.415 | 0.7339 |
| 1.00E+04 | | | |
| IM PDA001 1.00E+06, IM Vehicle | 26.602 | 26.415 | 0.485 |
| IM PDA001 1.00E+06, IV Vehicle | 19.580 | 26.415 | 0.1418 |
| IV PDA001 1.00E+04, IM Vehicle | 22.130 | 26.415 | 0.0981 |
| IV PDA001 1.00E+04, IV Vehicle | 15.108 | 26.415 | 0.2543 |
| IM Vehicle, IV Vehicle | −7.022 | 26.415 | 0.5939 |

### 2. Clinical Observation

There were no differences in clinical scores between groups

TABLE 10
Clinical Scores
Clinical Observation

| Treatment | | | | | | |
|---|---|---|---|---|---|---|
| Group | Day 1 | Day 7 | Day 14 | Day 21 | Day 28 | Day 34 |
| B | 1.00 ± 0.00 | 0.83 ± 0.17 | 1.00 ± 0.00 | 0.83 ± 0.17 | 0.67 ± 0.21 | 0.33 ± 0.21 |
| D | 1.00 ± 0.00 | 1.00 ± 0.00 | 1.17 ± 0.17 | 1.00 ± 0.00 | 0.83 ± 0.31 | 0.33 ± 0.21 |
| E | 1.00 ± 0.00 | 1.17 ± 0.17 | 1.33 ± 0.21 | 1.00 ± 0.26 | 0.50 ± 0.22 | 0.17 ± 0.17 |
| F | 1.00 ± 0.00 | 1.00 ± 0.00 | 1.00 ± 0.00 | 0.50 ± 0.22 | 0.33 ± 0.21 | 0.17 ± 0.17 |
| H | 1.00 ± 0.00 | 1.00 ± 0.00 | 0.83 ± 0.17 | 0.83 ± 0.31 | 1.00 ± 0.37 | 0.67 ± 0.33 |

### Conclusions

Hind limb ischemia was produced in mature, male Sprague-Dawley rats by occlusion of the left femoral artery. Cells were delivered by IV infusion or IM injection at Day 1 after HLI. At Day 34 after HLI, blood flow scores tended to be increased in IV PDA-002 1.00E+04, and IM PDA-002 1.00E+06-treated cell groups compared to appropriate control groups.

The present disclosure is not to be limited in scope by the specific embodiments described herein. Indeed, various modifications of the disclosure in addition to those described herein will become apparent to those skilled in the art from the foregoing description and the accompanying figures. Such modifications are intended to fall within the scope of the appended claims.

What is claimed is:

1. A method for modulating Glucagon-Like Peptide 1 (GLP-1) agonist induced muscle loss in a subject being treated with a GLP-1 agonist, comprising administrating to the subject a therapeutically effective amount of a placenta derived biological material having a continuous lipid bilayer membrane, an umbilical cord derived biological material having a continuous lipid bilayer membrane, or a combination thereof.

2. The method of claim 1, wherein modulating the GLP-1 agonist induced muscle loss in the subject comprises: (a) retarding the GLP-1 agonist induced muscle loss; (b) arresting the GLP-1 agonist induced muscle loss; or (c) ameliorating the GLP-1 agonist induced muscle loss.

3. The method of claim 1, wherein the biological material having a continuous lipid bilayer comprises; (a) a population of placenta derived adherent cells; (b) secretomes isolated from placenta; (ii) umbilical cord; or (iii) a combination of (i) and (ii); (b) exosomes secreted from cells of a population of placenta derived adherent cells; or (c) a combination of (a) and (b).

4. The method of claim 3, wherein the secretomes isolated from placenta contain exosomes.

5. The method of claim 3, wherein the secretion of the exosomes from the placenta derived adherent cells occurs (a) in vivo, (b) in vitro, (c) ex vivo, or (d) any combination of (a)-(c).

6. The method of claim 1, wherein administration of the biological material having a continuous lipid bilayer to the subject is selected from the group consisting of intradermal administration, subcutaneous administration, intramuscular administration, and intravenous administration.

7. The method of claim 6, wherein administration of the biological material having a continuous lipid bilayer is intramuscular administration.

8. The method of claim 1, wherein the placenta derived adherent cells comprise: (a) PDA-001 cells; (b) PDA-002 cells; or (c) a combination of (a) and (b).

9. The method of claim 1, wherein the GLP-1 agonist comprises: (a) dulaglutide; (b) exenatide; (c) exenatide extended-release; (d) liraglutide; (e) dulaglutide; (f) semaglutide injection; (g) semagulutide tablet; (h) lixisenatide; (i) albiglutide; (j) tirzepatide; or (k) efpeglenatide.

10. The method of claim 1, wherein the subject is being treated with a GLP-1 agonist for:
(a) the prevention, treatment or prevention and treatment of diabetes;
(b) the delaying or preventing of diabetic disease progression;
(c) the prevention, treatment or prevention and treatment of eating disorders; or
(d) any combination of (a)-(d).

11. A method for modulating GLP-1 agonist induced muscle loss in a subject being treated with a GLP-1 agonist, comprising the administration to the subject of a therapeutically effective amount of:
(a) a population of placenta derived adherent cells;
(b) exosomes secreted from placenta derived adherent cells;
(c) secretomes isolated from placenta; or
(d) any combination of (a)-(c).

12. The method of claim 11, wherein the secretomes isolated from placenta contain exosomes.

13. The method of claim 11, wherein modulating the GLP-1 agonist induced muscle loss in the subject comprises: (a) retarding the GLP-1 induced muscle loss; (b) arresting the GLP-1 induced muscle loss; or (c) ameliorating the GLP-1 agonist induced muscle loss.

14. The method of claim 11, wherein the subject is being treated with the GLP-1 agonist for: (a) type II diabetes; (b) weight loss; or (c) a combination of (a) and (b).

15. The method of claim 11, wherein the administration is selected from the group consisting of intradermal administration, subcutaneous administration, intramuscular administration, and intravenous administration.

16. The method of claim 15, wherein administration is intramuscular administration.

17. The method of claim 11, wherein the placenta derived adherent cells comprise: (a) PDA-001 cells; (b) PDA-002 cells; or (c) a combination of (a) and (b).

18. The method of claim 11, wherein the GLP-1 agonist comprises: (a) dulaglutide; (b) exenatide; (c) exenatide extended-release; (d) liraglutide; (e) dulaglutide; (f) semaglutide injection; (g) semagulutide tablet; (h) lixisenatide; (i) albiglutide; (j) tirzepatide; or (k) efpeglenatide.
